(19) United States
(12) Patent Application Publication
(10) Pub. No.: US 2026/0348920 A1
(43) Pub. Date: Nov. 5, 2026

(54) ULTRASOUND IMAGING DEVICE

(71) Applicant: MODULEUS, TOURS (FR)

(72) Inventors: Edgard JEANNE, TOURS (FR); Mathieu ROY, TOURS (FR)

(21) Appl. No.: 19/168,572

(22) PCT Filed: Mar. 11, 2024
(86) PCT No.: PCT/EP2024/056414

§ 371 Date: Sep. 24, 2025

(30) Foreign Application Priority Data
Mar. 30, 2023 (FR) FR2303100

(51) Int. Cl. G01N 29/06; A61B 8/00; A61B 8/08; G01N 29/28

(52) U.S. Cl. CPC G01N 29/0654; A61B 8/0891; A61B 8/4281; G01N 29/28; G01N 2291/106

(57) ABSTRACT

The present description relates to an ultrasound imaging device (200) comprising a set (101) of ultrasound transducers, an acoustic coupling layer (105) coating said set (101) of ultrasound transducers and a suction system (203) adapted to exert a suction force tending to press an object to be imaged (111), disposed opposite the acoustic coupling layer (105), against an upper surface of the coupling layer (105).

This application is based on and claims priority from French patent application FR2303100 filed on Mar. 30, 2023, entitled “Ultrasound imaging device,” which is considered to be an integral part of this description within the limits provided by law.

### TECHNICAL FIELD

The present disclosure relates generally to the field of ultrasound imaging devices and more particularly, to devices for acquiring skin prints and/or microvascular imaging devices based on ultrasound transducers.

### BACKGROUND ART

An ultrasound imaging device typically comprises a plurality of ultrasound transducers and an electronic control circuit connected to the transducers. In operation, the set of transducers is positioned facing an object or body from which an image is to be acquired. The electronic control circuit is configured to apply electrical excitation signals to the transducers, causing the transducers to emit ultrasound waves towards the body to be analyzed. The ultrasound waves emitted by the transducers are reflected by the body to be analyzed (by its internal and/or superficial structure), then return to the transducers, which convert them back into electrical signals. These electrical response signals are read by the electronic control circuit and can be stored and analyzed to deduce information about the body being studied.

It would be desirable to improve, at least in part, certain aspects of known ultrasound imaging devices.

### SUMMARY OF INVENTION

To this end, one embodiment provides an ultrasound imaging device comprising a set of ultrasound transducers, an acoustic coupling layer coating the set of ultrasound transducers and a (motorized) suction system adapted to exert a suction force tending to press an object to be imaged, disposed opposite the acoustic coupling layer, against an upper surface of the coupling layer.

According to one embodiment, the device comprises a protective housing comprising an upper plate located above the acoustic coupling layer, such that a cavity separates a lower surface of the upper plate of the housing from the upper surface of the acoustic coupling layer.

According to one embodiment, the upper plate has a through-opening intended to be placed opposite the object to be imaged.

According to one embodiment, the through-opening is located opposite the set of ultrasound transducers.

According to one embodiment, the suction system is connected to said cavity by a sealed conduit.

According to one embodiment, the suction system is configured to apply an at least partial vacuum in the cavity during an ultrasound image acquisition phase, so as to press the object to be imaged against the upper surface of the acoustic coupling layer.

According to one embodiment, the acoustic coupling layer is made of a polymer material.

According to one embodiment, the suction system comprises a vacuum pump.

One embodiment provides for the use of an ultrasound imaging device as defined above, for acquiring biometric images.

One embodiment provides for the use of an ultrasound imaging device as defined above, for acquiring medical images.

One embodiment provides for the use of an ultrasound imaging device as defined above, for non-destructive ultrasound testing applications.

### BRIEF DESCRIPTION OF DRAWINGS

The foregoing features and advantages, as well as others, will be described in detail in the following description of specific embodiments given by way of illustration and not limitation with reference to the accompanying drawings, in which:

FIG. 1 is a cross-sectional view schematically representing an example of an ultrasound imaging device; and

FIGS. 2A and 2B are cross-sectional views schematically representing an example of an ultrasound imaging device according to one embodiment.

### DESCRIPTION OF EMBODIMENTS

Like features have been designated by like references in the various figures. In particular, the structural and/or functional features that are common among the various embodiments may have the same references and may dispose identical structural, dimensional and material properties.

For the sake of clarity, only the steps and elements that are useful for an understanding of the described embodiments have been illustrated and described in detail. In particular, the construction of the ultrasound transducers and electronic control circuits of the described devices has not been detailed, as the described embodiments are compatible with the usual constructions of these elements. Furthermore, the various applications of the described devices have not been detailed, as the described embodiments are compatible with all or most of the usual applications of ultrasound imaging devices, and in particular the applications for imaging parts of the human or animal body.

Unless indicated otherwise, when reference is made to two elements connected together, this signifies a direct connection without any intermediate elements other than conductors, and when reference is made to two elements coupled together, this signifies that these two elements can be connected or they can be coupled via one or more other elements.

In the following disclosure, unless indicated otherwise, when reference is made to absolute positional qualifiers, such as the terms “front”, “back”, “top”, “bottom”, “left”, “right”, etc., or to relative positional qualifiers, such as the terms “above”, “below”, “higher”, “lower”, etc., or to qualifiers of orientation, such as “horizontal”, “vertical”, etc., reference is made to the orientation shown in the figures.

Unless specified otherwise, the expressions “around”, “approximately”, “substantially” and “in the order of” signify within 10%, and preferably within 5%.

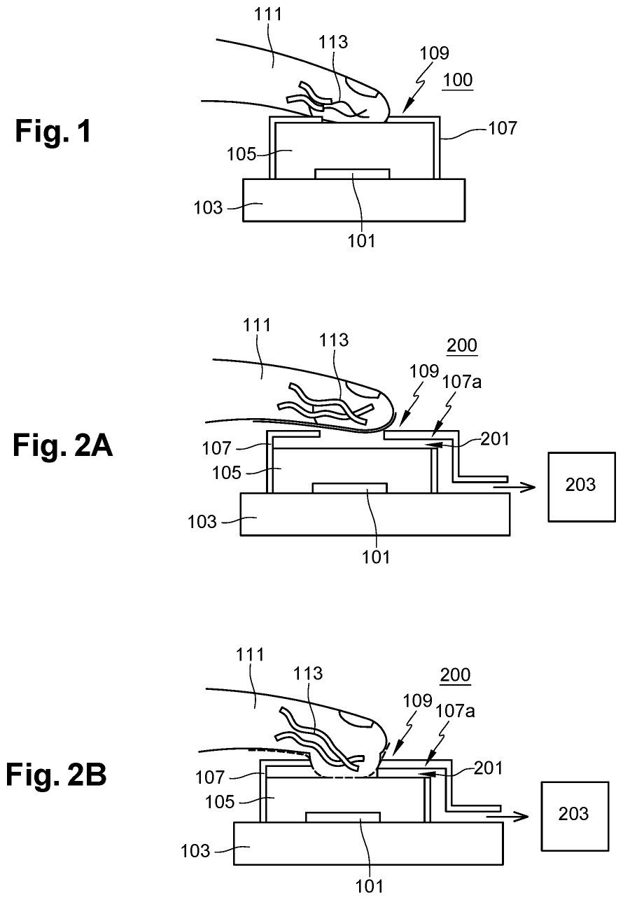

FIG. 1 is a cross-sectional view schematically representing an example of an ultrasound imaging device 100, for example for biometric applications.

The device 100 in FIG. 1 comprises a set 101 of ultrasound transducers (not shown in detail in the figure), for example arranged in a matrix, a bar or any other arrangement. The transducers in the set 101 are, for example, CMUT-type transducers (membrane capacitive ultrasound transducers), PMUT-type transducers (membrane piezoelectric transducers), crystal transducers or any other type of ultrasound transducers, for example piezoelectric or piezocomposite transducers or single crystal transducers.

The transducers of the set 101 are, for example, integrated into a monolithic chip, for example formed in and on a semiconductor substrate, for example a silicon substrate, or alternatively in and on an insulating substrate, for example a glass substrate (not shown in the figure).

In the example shown, the set 101 of transducers is mounted on a printed circuit board 103. The printed circuit board 103 comprises, for example, a support made of an electrically insulating material, such as plastic, and a set of metal interconnection pads and tracks (not shown in the figure) formed on at least one surface of the support. In this example, the set 101 of ultrasound transducers is secured and electrically connected to the upper surface of the printed circuit board 103.

The device 100 of FIG. 1 may further comprise one or more electronic circuits (not shown) for powering and controlling the set 101 of ultrasound transducers. The electronic power supply and control circuits are, for example, secured and electrically connected to one surface of the printed circuit board 103.

The electronic power supply and control circuits comprise, for example, emitting circuits adapted to supply electrical excitation signals to the ultrasound transducers so as to cause the transducers to emit ultrasound waves, and receiving circuits adapted to read electrical response signals generated by the ultrasound transducers of the set 101 under the effect of an ultrasound wave received from the object to be imaged.

The device 100 of FIG. 1 further comprises a coupling layer 105, for example made of a polymer material, for example electrically insulating, coating the upper surface of the set 101 of ultrasound transducers. For example, the layer 105 is disposed on and in contact with the upper surface of the set 101 of ultrasound transducers. Alternatively, the layer 105 is secured to the upper surface of the set 101 of transducers by means of an adhesive layer, not shown.

For example, the coupling layer 105 extends continuously over the entire upper surface of the set 101 of ultrasound transducers. In the example shown, the coupling layer also extends laterally beyond the edges of the set 101 of transducers, thus also covering the sides of the set 101.

The thickness of the coupling layer 105 is, for example, between 100 μm and 10 mm, for example between 500 μm and 2 mm.

The upper surface of the layer 105 defines a contact surface on which an object to be imaged is placed, for example one or more fingers of a user, the palm of a user or any other part of the human or animal body that is to be imaged.

The layer 105 has an ultrasound coupling function between the transducers of the set 101 and the object to be imaged. In particular, the layer 105 is preferably relatively flexible and deformable to avoid the presence of air gaps between the transducers of the set 101 and the object to be imaged. In addition, the layer 105 preferably has an acoustic impedance adapted to that of the object to be imaged, for example an acoustic impedance substantially equal to the acoustic impedance of the skin. Thus, the layer 105 allows to maximize the transfer of acoustic energy between the ultrasound transducers and the object to be analyzed.

The layer 105 may also have a protective function for the device and in particular, for the ultrasound transducers of the set 101. In particular, the layer 105 preferably has a relatively high mechanical strength so that it does not degrade over time and in contact with the objects to be imaged.

In the example shown in FIG. 1, the acoustic coupling layer 105 and the set 101 of transducers are encapsulated in a protective housing 107, for example made of a rigid material, for example a metallic material or an electrically insulating material of the plastic type. The protective housing 107 includes a through-opening or window 109 opposite the set of ultrasound transducers, allowing free access to the upper surface of the acoustic coupling layer 105.

In operation, the object to be imaged is placed in contact with the upper surface of the acoustic coupling layer 105 opposite the opening 109.

In the example shown in FIG. 1, the object to be imaged is a finger 111. The opening 109 has, for example, substantially the same dimensions as those of a part of the finger, for example a fingertip, which is to be imaged.

In practice, to avoid the presence of an air gap between the surface of the finger and the coupling layer 105, and to limit the risk of finger movement, users tend to apply relatively high pressure to the coupling layer 105 with their finger throughout the duration of an ultrasound image acquisition phase.

Experiments conducted by the inventors have shown that, for certain applications, such as microvascular imaging applications, the quality of the images acquired may be degraded due to the pressure exerted by the user during acquisition. For example, imaging of low blood flow and microscopic vessels 113 located at the tips of the fingers and toes is difficult to achieve because these vessels may undergo compression, resulting in decreased blood flow.

FIGS. 2A and 2B are cross-sectional views schematically representing an example of an ultrasound imaging device 200 according to one embodiment.

The device 200 includes elements common to the device 100 of FIG. 1. These elements will not be described in detail again. Only the differences from the device 100 of FIG. 1 will be described in detail below.

The device 200 differs from the device 100 in FIG. 1 mainly in that it comprises a motorized suction system adapted to exert a suction force tending to press the object to be imaged against the upper surface of the coupling layer 105. Thus, the user himself/herself does not have to exert a manual pressure to press the object to be imaged against the surface of the coupling layer 105. This allows to limit the risk of excessive compression of the object, which can degrade the quality of the ultrasound images acquired. In particular, for microvascular imaging applications, this allows to prevent an unwanted compression of the vessels to be imaged.

In the example of FIGS. 2A and 2B, the protective housing 107 includes an upper plate 107a disposed above the upper surface of the acoustic coupling layer 105. In this example, the housing 107 is arranged so that an air gap or a cavity 201 separates the upper surface of the acoustic coupling layer 105 from the lower surface of the upper plate 107a of the housing.

The air gap or cavity 201 may have a relatively small thickness, for example between 0.1 mm and 10 mm, for example between 0.5 mm and 5 mm.

When the user places his/her finger on the device, it rests on and comes into contact with the upper surface of the plate 107a. A central part of the finger, corresponding to the part to be imaged, closes the opening 109 and is thus opposite the upper surface of the coupling layer 105, separated from the upper surface of the layer 105 only by the cavity 201.

The air gap or cavity 201 is connected to a suction system 203 (not shown in the figure) via a sealed conduit (not shown in the figure). For example, the air gap or cavity 201 is connected to the atmosphere outside the housing 107 solely via the opening 109 and the suction system 203.

The suction system 203 is, for example, a vacuum pump or any other motorized suction system. The provision of an air gap of relatively small thickness advantageously allows to limit the space required for the suction system.

FIG. 2A illustrates a configuration in which the motorized suction system 203 is inactive. In this case, the finger rests on the upper plate 107a of the protective housing, with the part of the finger to be imaged completely or partially closing the opening 109. It should be noted that, in this configuration, the part of the finger to be imaged is not in contact with the upper surface of the acoustic coupling layer 105 but is separated from it by the air gap 201. Due to the presence of an air gap between the finger and the acoustic coupling layer 105, this configuration is not favorable to the acquisition of an ultrasound image.

FIG. 2B illustrates a configuration in which the motorized suction system 203 is active. In this case, an at least partial vacuum is created in the cavity 201, causing the part of the finger to be imaged to be sucked in the interior of the cavity. Under the effect of the suction, the part of the finger to be imaged is pressed against the upper surface of the acoustic coupling layer 105. An ultrasound image of the finger can then be acquired.

The suction applied during the acquisition phase has several advantages.

In particular, it allows the finger, or more generally any object or organ to be imaged, to be held in a position opposite the set 101 of ultrasound transducers, in contact with the upper surface of the coupling layer 105, via a suction force automatically calibrated and controlled by a motorized system.

This allows, for example, to hold the sensor in place autonomously during the acquisition, without having to provide a specific human or mechanical holding system.

This also allows to prevent micro-movements of the area to be imaged, which could degrade the quality of the acquired images.

Furthermore, this allows to prevent the risk of excessive manual pressure being applied by the user, which could deform or compress the object to be imaged and thus degrade the quality of the acquired image.

In the case of a microvascular imaging application, one advantage is to prevent the blood vessels in the part of the finger to be imaged from undergoing compression that could impair the quality of the acquired image. On the contrary, suction can cause localized dilation in the part of the finger to be imaged, improving image quality.

Compared to the device 100 in FIG. 1, the proposed solution also allows to increase the contact surface between the finger and the acoustic coupling layer 105.

Various embodiments and variants have been described. Those skilled in the art will understand that certain features of these various embodiments and variants could be combined, and other variants will be apparent to those skilled in the art. In particular, the embodiments described are not limited to the example of arrangement of the suction system 203 and the protective housing 107 of the device shown in FIGS. 2A and 2B. More generally, those skilled in the art will know how to adapt the arrangement of the suction system 203 and the protective housing 107 according to the application in question and the type of object to be imaged.

Furthermore, the embodiments described are not limited to the above-mentioned example of application to microvascular imaging devices. As a variant, the proposed suction system can be applied to fingerprint acquisition devices to facilitate positioning and holding in position of the finger during acquisition.

Furthermore, the embodiments described are not limited to biometric applications but can be adapted to any ultrasound imaging system for parts of the human or animal body, for example for medical applications.

In addition, the embodiments described may be applied to ultrasound imaging devices for non-living objects, for example for non-destructive ultrasound testing applications. The provision of the motorized suction system then allows to facilitate positioning and holding in position of the acquisition device opposite the object to be imaged.

Finally, the practical implementation of the embodiments and variants described herein is within the capabilities of those skilled in the art based on the functional description provided hereinabove.

1. An ultrasound imaging device comprising a set of ultrasound transducers, an acoustic coupling layer coating said set of ultrasound transducers and a motorized suction system adapted to exert a suction force tending to press an object to be imaged, disposed opposite the acoustic coupling layer, against an upper surface of the coupling layer.

2. The ultrasound imaging device according to claim 1, comprising a protective housing comprising an upper plate located above the acoustic coupling layer, such that a cavity separates a lower surface of the upper plate of the housing from the upper surface of the acoustic coupling layer.

3. The ultrasound imaging device according to claim 2, wherein said upper plate has a through-opening intended to be placed opposite the object to be imaged.

4. The ultrasound imaging device according to claim 3, wherein said through-opening is located opposite the set of ultrasound transducers.

5. The ultrasound imaging device according to claim 2, wherein the suction system is connected to said cavity by a sealed conduit.

6. The ultrasound imaging device according to claim 2, wherein the suction system is configured to apply an at least partial vacuum in the cavity during an ultrasound image acquisition phase, so as to press the object to be imaged against the upper surface of the acoustic coupling layer.

7. The ultrasound imaging device according to claim 1, wherein the acoustic coupling layer is made of a polymer material.

8. The ultrasound imaging device according to claim 1 wherein the suction system comprises a vacuum pump.

9. A use of an ultrasound imaging device according to claim 1, for acquiring biometric images.

10. The use of an ultrasound imaging device according to claim 1, for acquiring medical images.

11. The use of an ultrasound imaging device according to claim 1, for non-destructive ultrasound testing applications.
